(19) United States
(12) Patent Application Publication
(10) Pub. No.: US 2013/0035738 A1
(43) Pub. Date: Feb. 7, 2013

(54) METHODS AND SYSTEMS FOR DETERMINING PACING PARAMETERS BASED ON REPOLARIZATION INDEX

(71) Applicants: Edward Karst, South Pasadena, CA (US); Kyungmoo Ryu, Palmdale, CA (US); Stuart Rosenberg, Castaic, CA (US); Allen Keel, San Francisco, CA (US)

(72) Inventors: Edward Karst, South Pasadena, CA (US); Kyungmoo Ryu, Palmdale, CA (US); Stuart Rosenberg, Castaic, CA (US); Allen Keel, San Francisco, CA (US)

(73) Assignee: PACESETTER, INC., Sylmar, CA (US)

(21) Appl. No.: 13/197,665

(22) Filed: Aug. 3, 2011

(51) Int. Cl. A61N 1/368

(52) U.S. Cl. 607/25

(57) ABSTRACT

Methods and systems are provided for determining pacing parameters for an implantable medical device (IMD). The methods and systems provide electrodes in the right atrium (RA), right ventricle (RV) and left ventricle (LV). The methods and systems sense RV cardiac signals and LV cardiac signals at an RV electrode and an LV electrode, respectively, over multiple cardiac cycles, to collect global activation information. The methods and systems identify a T-wave in the LV cardiac signal. The methods and systems calculate a repolarization index based at least in part on a timing of the T-wave identified in the LV cardiac signal. The methods and systems set at least one pacing parameter based on the repolarization index, wherein the at least one pacing parameter that is set represents at least one of an AV delay, an inter-ventricular interval and an intra-ventricular interval. Optionally, the methods and systems may deliver an RV pacing stimulus at the RV electrode such that the LV cardiac signal sensed thereafter includes the RV pacing stimulus followed by a T-wave. The methods and systems determine a waveform metric such as at least one of a QT interval, T-wave duration, and T-wave amplitude, and utilize the waveform metric to determine as the repolarization index.

### FIELD OF THE INVENTION

Embodiments of the subject matter disclosed herein generally relate to methods and systems for treating cardiac dysfunction with pacemakers and cardiac resynchronization therapy (CRT).

### BACKGROUND OF THE INVENTION

The heart is a muscular organ comprising multiple chambers that operate in concert to circulate blood throughout the body's circulatory system. The contractions of the muscular walls of each chamber of the heart are controlled by a complex conduction system that propagates electrical signals to the heart muscle to effectuate the atrial and ventricular contractions necessary to circulate the blood.

Electrical impulses are normally initiated in the right atrium at the sinoatrial (SA) node and propagate to the ventricles through the atrioventricular (AV) node. The SA node initiates an electrical signal that spreads through the muscle of the right and left atria and the atrioventricular node. As a result, the right and left atria contract to pump blood into the right and left ventricles, respectively. At the atrioventricular node, conduction of the electrical signal slows before propagating through the right and left ventricles. Within the right and left ventricles the conduction system includes right and left bundles branches that extend from the atrioventricular node via the Bundle of His. The electrical impulse spreads through the heart muscle of the right and left ventricles. As a result, the right and left ventricles contract to pump blood throughout the body. Normally, the ventricles of the heart contract synchronously to circulate blood to the systemic circulation and the lungs.

When areas of the heart tissue experience an unhealthy state, a delay may be introduced into the electrical signal transmission. When the electrical signal to the left ventricle is delayed, the right ventricle begins to contract before the left ventricle, instead of contracting simultaneously. The delay in contraction, between right ventricle and left ventricle, may result in an asynchronous or uncoordinated contraction of the ventricles and a mis-timing in the contraction pattern of the atria and ventricles. Other conduction abnormalities may contribute to unsynchronized and less efficient contraction of the heart. The abnormalities further reduce the pumping ability of the heart muscle.

During the cardiac cycle, individual chambers of the heart alternately progress through a systolic phase and then a diastolic phase. During the systolic phase, the heart tissue of a corresponding chamber undergoes a depolarization of cellular transmembrane potential and during a diastolic phase the heart tissue of the same chamber undergoes a repolarization of transmembrane potential. Depolarization of a mass of cardiac myocytes generates an action potential and leads to mechanical contraction of the tissue.

CRT and pacemaker devices can be used to improve the conduction pattern and sequence of the heart. CRT and pacemaker devices involve the use of an artificial electrical stimulator that is surgically implanted within the patient's body. The artificial electrical stimulator may have multiple electrodes. The electrodes can be placed at a desired location proximate to the heart. The artificial electrical stimulator sends electrical impulses to the heart, via the electrodes, to effectuate synchronous atrial and/or ventricular contractions. Various conventional processes exist to determine CRT and pacing parameters, such as the AV delay and the V-V interval. At least one conventional process derives the AV delay and the V-V interval based on intrinsic activation time, which reflects time of depolarization of a mass of cardiac cells. This process recommends AV delay based on intrinsic PR interval, less a correction factor, and suggests V-V interval based on sensed and paced inter-ventricular conduction delays. These techniques use the P and R waves, which are measurements indicating cardiac depolarization.

However, there exists a desire to continue to improve upon the usefulness of the CRT and pacemaker devices. For example, conventional processes that derive pacing parameters based on information from depolarization do not entirely account for conduction abnormalities that may be present in some patients. Lines of conduction block and transmural heterogeneity of action potential propagation may lead to variation in depolarization, and there may be varying delay in electromechanical activation within the tissue.

### SUMMARY

In accordance with embodiments, a method and system are provided for determining pacing parameters for an implantable device (IMD). The method and system provide leads containing electrodes in the right atrium (RA), right ventricle (RV) and left ventricle (LV), which are used for sensing RA, RV cardiac signals and LV cardiac signals at the RA, RV electrodes and LV electrodes, respectively. One embodiment uses an LV lead containing four electrodes, which are used to sense the cardiac electrical activity at four locations in the left heart. The sensing of the cardiac signals may be done over a single cardiac cycle or over multiple cardiac cycles, so as to collect activation information. Next, the method and system involve identifying a T-wave in the cardiac signal. The method and system use the timing of the T-wave, at least in part, to calculate a repolarization index. The method and system set at least one of the pacing parameters based on the repolarization index. The pacing parameter that is set may be for Atrio-Ventricular (AV) delay, inter-ventricular interval and/or intra-ventricular interval.

Optionally, the method and system may further comprise delivering RV pacing stimulus at the RV electrode such that the LV cardiac signal sensed thereafter includes a response to the RV pacing stimulus followed by a T-wave. The method and system may determine at least one waveform metric such as a QT interval, T-wave duration, and T-wave amplitude at each LV electrode, and utilize the waveform metric at the various electrodes to calculate the repolarization index. The delivering and determining operations are repeated for multiple cardiac cycles to acquire the waveform metric at each electrode combination, and may further comprise adjusting an AV delay between the multiple cardiac cycles, collecting QT intervals at each electrode combination for the multiple cardiac cycles; and determining a dispersion of the QT interval at each electrode combination for the multiple cardiac cycles.

Alternatively, the method and system for determining pacing parameters for the IMD may further comprise measuring times of occurrence of T-waves in the LV cardiac signal for each of one or more electrode configurations and in the RV cardiac signal, and determining a difference between the times of occurrence. The repolarization index may be calculated at least in part based on the difference between the times of occurrence.

Alternatively, the method and system for determining pacing parameters for an IMD may deliver an RV pacing stimulus at the RV electrode, perform the sensing operation after delivering the RV pacing stimulus, identify a T-wave in the RV cardiac signal, and calculate a ventricular conduction delay (IVCD_RVpace) as a time between occurrence of the T-wave in the RV cardiac signal and a time of occurrence of the T-wave in an LV cardiac signal. The IVCD_RVpace is used to calculate the repolarization index to then set at least one pacing parameter.

Optionally, the method and system may deliver an LV pacing stimulus at an LV electrode, perform the sensing operation after delivering the LV pacing stimulus, identify a T-wave in the RV cardiac signal, and calculate a ventricular conduction delay (IVCD_LVpace) as a time between occurrence of the T-wave in the RV cardiac signal and a time of occurrence of the T-wave in the LV cardiac signal. The IVCD_LVpace is used to calculate the repolarization index to then set the at least one pacing parameter.

Optionally, the method and system may deliver an LV pacing stimulus at an LV electrode and an RV pacing stimulus at the RV electrode during different cardiac cycles, and calculating first and second ventricular conduction delays based on the LV and RV pacing stimuli. The first and second ventricular conduction delays are used to calculate the repolarization index to then set the at least one pacing parameter.

Optionally, the inter- or intra-ventricular interval may be set based on the repolarization index. Alternatively, multiple LV electrodes may be provided in or proximate to the LV, and the method switches between different RV_LV combinations utilizing the RV electrode and different ones of the LV electrodes. The method repeats the sensing, identifying and calculating operations for the different RV_LV combinations. Optionally, the method and system may identify one of the RV_LV combinations that have a longest time between an occurrence of the T-wave in the RV cardiac signal and a time of occurrence of the T-wave in the LV cardiac signal, and utilize the identified one of the RV_LV combinations when performing an AV delay extension test.

Optionally, the method and system may switching between different RV_LV combinations utilizing the RV electrode and the LV electrodes, and delivering LV pacing stimuli at the LV electrodes during different cardiac cycles. Additionally, the method may involve detecting R-waves in the RV cardiac signals sensed at the RV electrode. The sensing may be performed for each of the LV pacing stimuli from the corresponding LV electrodes. Further, the method may involve identifying one of the RV_LV combinations that has a shortest time between an occurrence of the R-wave in the RV cardiac signal and a time of occurrence of the corresponding LV pacing stimulus.

### BRIEF DESCRIPTION OF THE DRAWINGS

The drawings illustrate generally, by way of example, but not by way of limitation, various embodiments discussed in the present document.

FIG. 1 illustrates an implantable medical device (IMD) coupled to a heart in accordance with an embodiment.

FIG. 2 illustrates a block diagram of exemplary internal components of the IMD shown in FIG. 1.

FIG. 3 illustrates a process to collect waveform metrics utilizing a T-wave in accordance with an embodiment.

FIG. 4 illustrates a process to determine whether a current AV delay has reached an AV delay limit in accordance with an embodiment.

FIG. 5 illustrates a process to collect interval related information following a ventricular paced event in accordance with an embodiment.

FIG. 6 illustrates a process to determine an intra-ventricular conduction delay from the RV to the LV in accordance with an embodiment.

FIG. 7 illustrates a process to calculate intra-ventricular conduction delay following an LV pacing stimulus and a process to calculate a V-V interval in accordance with an embodiment.

FIG. 8 illustrates an alternate process to calculate an inter-ventricular interval based on the occurrence of a T-wave and an R-wave in accordance with an embodiment.

FIG. 9 illustrates an alternate process to perform an AV delay test utilizing a multipolar LV lead in accordance with an embodiment.

FIG. 10 illustrates an RA sensed and RV IEGM waveforms, along with markers in accordance with an embodiment.

FIG. 11 illustrates electrograms using a quadpolar LV lead in accordance with an embodiment.

FIG. 12 illustrates a distributed processing system in accordance with an embodiment.

### DETAILED DESCRIPTION

In the following detailed description, reference is made to the accompanying drawings which form a part hereof, and in which are shown by way of illustration specific embodiments in which the subject matter disclosed herein may be practiced. These embodiments, which are also referred to herein as “examples,” are described in sufficient detail to enable those skilled in the art to practice the subject matter disclosed herein. It is to be understood that the embodiments may be combined or that other embodiments may be utilized, and that structural, logical, and electrical variations may be made without departing from the scope of the subject matter disclosed herein. The following detailed description is, therefore, not to be taken in a limiting sense, and the scope of the subject matter disclosed herein is defined by the appended claims and their equivalents. In the description that follows, like numerals or reference designators will be used to refer to like parts or elements throughout. In the subject matter disclosed herein, the terms “a” or “an” are used, as is common in patent documents, to include one or more than one. In the subject matter disclosed herein, the term “or” is used to refer to a nonexclusive or, unless otherwise indicated. In the subject matter disclosed herein, the term “original settings” refers to the settings of an IMD prior to a medical procedure. As used throughout, the term “T-wave” may be in response to paced depolarization or spontaneous/intrinsic depolarization.

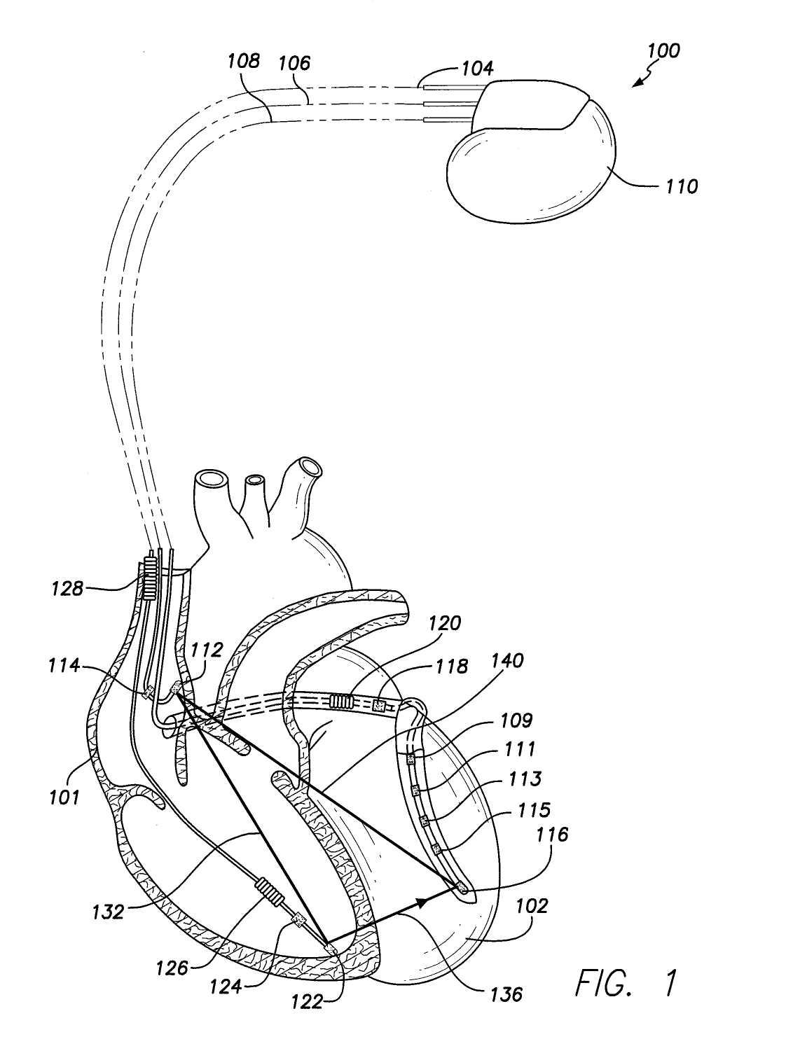

FIG. 1 illustrates an implantable medical device (IMD) coupled to a heart with electrodes in RA, RV, and LV in accordance with an embodiment. The IMD 100 may be a cardiac pacemaker, an ICD, a defibrillator, an ICD coupled with a pacemaker, a cardiac resynchronization therapy (CRT) pacemaker, a cardiac resynchronization therapy defibrillator (CRT-D), and the like. The IMD 100 includes a housing 110 that is joined to leads 104, 106, 108. The leads 104, 106, 108 are located at various locations of the heart 102, such as an atrium, a ventricle, or both, to measure cardiac signals of the heart 102. The leads 104, 106, 108 include the right ventricular (RV) lead 104, the right atrial (RA) lead 106, and the coronary sinus (CS)-based left ventricular (LV) lead 108. Electrodes are provided on leads 104, 106, 108 for sensing cardiac signals and/or for delivering stimulus or stimulation pulses to the heart 102. The housing 110 may be one of the electrodes and is often referred to as the “can”, “case”, or “case electrode.”

The RV lead 104 includes an RV tip electrode 122, an RV ring electrode 124 and may include an RV coil electrode 126. The RV lead 104 may include a superior vena cava (SVC) coil electrode 128. The right atrial lead 106 includes an atrial tip electrode 112 and an atrial ring electrode 114. The coronary sinus lead 108 includes a left ventricular (LV) tip electrode 116, a left atrial (LA) ring electrode 118 and an LA coil electrode 120. Alternatively, the coronary sinus lead 108 may be a quadripolar or multipolar lead that includes multiple electrodes 109, 111, 113, 115 disposed within the left ventricle. Leads and electrodes other than those shown in FIG. 1 may be included in the IMD 100 and positioned in or proximate to the heart 102.

The IMD 100 monitors cardiac signals of the heart 102 to determine if and when to deliver stimulus pulses to one or more chambers of the heart 102. The IMD 100 may deliver pacing stimulus pulses to pace the heart 102 and maintain a desired heart rate and/or shocking stimulus pulses to treat an abnormal heart rate such as tachycardia or bradycardia.

FIG. 1 also further displays optional position of RV lead 104 at 122, RA lead 106 at 112, and LV lead 108 at 116. The IMD 100 uses cathode tip electrodes (RA 112, RV 122, LV 116) for sensing the intrinsic cardiac activity and pacing by providing the minimum amount of energy (both volts and pulse width) required to reliably depolarize the cardiac chamber. These leads provide sensed electrogram waveforms from three chambers: RA, RV and LV. Further, the lines 132, 136, and 140 represent the vector between RA, RV and LV. These cardiac vectors may appear and disappear three times, namely during atrial depolarization, during ventricular depolarization, and during ventricular repolarization. The deflection of the electrocardiograph from ground is proportional to the component size of the cardiac vector in the direction of the lead. These measurements are used to calculate optimized cardiac pacing.

Pacing stimulus is delivered to an electrode pair, which is typically a set of two electrodes on a single lead (bipolar) but can also be a cathode on the lead and an anode at a remote location (unipolar, when the anode may be on another lead or may be the device can). Sensing is typically accomplished by measuring the analog electrical signal across a pair of electrodes. Typically the electrode pair is on a single lead (bipolar), or the anode may be remote (a unipolar signal, which may use the device can as anode).

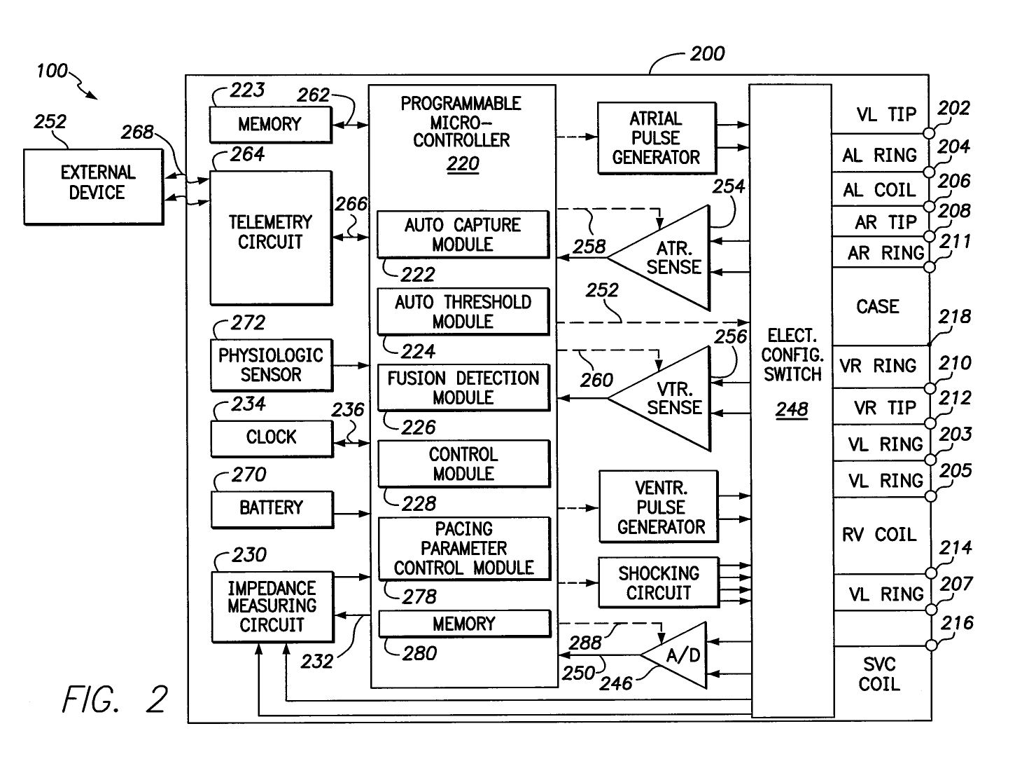

FIG. 2 illustrates a block diagram of exemplary internal components of the IMD 100 shown in FIG. 1 to provide optimized pacing to a heat in accordance with an embodiment. The IMD 100 includes the housing 200 that includes a left ventricle tip input terminal (V_{L} TIP) 202, a left atrial ring input terminal (A_{L} RING) 204, a left atrial coil input terminal (A_{L} COIL) 206, a right atrial tip input terminal (A_{R} TIP) 208, a right ventricular ring input terminal (V_{R} RING) 210, a right ventricular tip input terminal (V_{R} TIP) 212, an RV coil input terminal 214, an SVC coil input terminal 216, a right atrial ring (A_{R} RING) terminal 211, and three left ventricular ring (V_{L} RING) terminals 203, 205 and 207. A case input terminal 218 may be coupled with the housing 200 of the IMD 100. The input terminals 202-218 may be electrically coupled with the electrodes 112-128 (shown in FIG. 1).

The IMD 100 includes a programmable microcontroller 220, which controls the operation of the IMD 100. The microcontroller 220 (also referred to herein as a processor, processor module, or unit) typically includes a microprocessor, or equivalent control circuitry, and may be specifically designed for controlling the delivery of stimulation therapy and may further include RAM or ROM memory, logic and timing circuitry, state machine circuitry, and I/O circuitry. The microcontroller 220 may include one or more modules and processors configured to perform one or more of the operations described above in connection with the FIG. 1.

An autocapture module 222 senses evoked responses of the heart 102 (shown in FIG. 1) in response to delivery of stimulus pulses to the heart 102 when the IMD 100 operates in the autocapture mode described above. For example, the autocapture module 222 may examine the waveforms of cardiac signals sensed after supplying a stimulus pulse to an atrium of the heart 101. The autocapture module 222 may increase the electrical potential of stimulus pulses in subsequent cardiac cycles when an evoked response of a current cardiac cycle indicates a loss of capture, as described above.

An autothreshold module 224 performs threshold searches when the IMD 100 operates in the autothreshold mode described above. For example, the autothreshold module 224 may incrementally decrease the electrical potential of stimulus pulses delivered to myocardium of the heart 102 (shown in FIG. 1) until a loss of capture is detected in a first predetermined number of consecutive cardiac cycles. The autothreshold module 224 then may incrementally increase the electrical potential of the stimulus pulses until capture is detected in a second predetermined number of consecutive cardiac cycles. In an embodiment, the first and second predetermined numbers of consecutive cardiac cycles is two, although the first and second predetermined numbers of cardiac cycles may differ in another embodiment.

A fusion detection module 226 identifies fusion-based behavior in myocardium of the heart 102 (shown in FIG. 1). In an embodiment, the fusion detection module 226 tracks the fusion beat count and the total beat count during the time period that the IMD 100 operates in each of the autocapture and autothreshold modes. A control module 228 automatically switches the IMD 100 between the autothreshold and autocapture modes based on the presence of fusion-based behavior detected by the fusion detection module 226.

A pacing control module (PCM) 278, designed specifically to interface with the RA, RV, and LV electrodes for sensing and pacing of cardiac chamber. The pacing optimization control module may perform repolarization and depolarization measurement to calculate, inter alia, AV timing optimization, V-V timing optimization, T-wave, multisite CRT AV timing optimization, multisite CRT V-V timing optimization, Global dispersion of repolarization, and multisite CRT pacing site selection. Further, the PCM 278 may be programmed for generating optimizes pacing based on global activation and repolarization, reflecting overall myocardial properties. Optionally, the PCM 278 may also be programmed to correct for transmural and LV endocardial conduction anomalies. The PCM 278 may perform pacing optimization and correction of transmural and LV endocardial by a pacing one or more sites in a multi-polar electrodes. Alternatively, the PCM 278 may be programmed to perform reducing activation/repolarization dispersion as a basis for more optimal target for device-based optimization.

The microprocessor 220 receives signals from the electrodes 109-128 (shown in FIG. 1) via an analog-to-digital (A/D) data acquisition system 246. The cardiac signals are sensed by the electrodes 109-128 and communicated to the data acquisition system 246. The cardiac signals are communicated through the input terminals 202-216 to an electronically configured switch bank, or switch, 248 before being received by the data acquisition system 246. The data acquisition system 246 converts the raw analog data of the signals obtained by the electrodes 109-128 into digital signals 250 and communicates the signals 250 to the microcontroller 220. A control signal 288 from the microcontroller 220 determines when the data acquisition system 246 acquires signals, stores the signals 250 in the memory 280, or transmits data to an external device 252.

The switch 248 includes a plurality of switches for connecting the desired electrodes 109-128 (shown in FIG. 1) and input terminals 202-218 to the appropriate I/O circuits. The switch 248 closes and opens switches to provide electrically conductive paths between the circuitry of the IMD 100 and the input terminals 202-218 in response to a control signal 282. An atrial sensing circuit 254 and a ventricular sensing circuit 256 may be selectively coupled to the leads 104-108 (shown in FIG. 1) of the IMD 100 through the switch 248 for detecting the presence of cardiac activity in the chambers of the heart 102 (shown in FIG. 1). The sensing circuits 254, 256 may sense the cardiac signals that are analyzed by the microcontroller 220. Control signals 258, 260 from the microcontroller 220 direct output of the sensing circuits 254, 256 that are connected to the microcontroller 220. An impedance measuring circuit 230 is enabled by the microcontroller 220 via a control signal 232. The impedance measuring circuit 230 may be electrically coupled to the switch 248 so that an impedance vector between any desired pairs of electrodes 109-128 may be obtained. The IMD 100 additionally includes a battery 270 that provides operating power to the circuits shown within the housing 200, including the microcontroller 220. The IMD 100 includes a physiologic sensor 272 that may be used to adjust pacing stimulation rate according to the exercise state of the patient.

A clock 234 may measure time relative to the cardiac cycles or cardiac signal waveforms of the heart 102 (shown in FIG. 1). The clock 234 measures elapsed amounts of time based on start and stop control signals 236 from the microcontroller 220 to determine the ventricular and atrial heart rates. Additionally, the clock 234 may track the amount of time elapsed between threshold searches. The elapsed time may be compared to a predetermined time period to determine whether to perform another threshold search, as described above.

The memory 223 may be embodied in a computer-readable storage medium such as a ROM, RAM, flash memory, or other type of memory. The microcontroller 220 is coupled to the memory 223 by a suitable data/address bus 262. The memory 223 may store programmable operating parameters and thresholds used by the microcontroller 220, as required, in order to customize the operation of IMD 100 to suit the needs of a particular patient. For example, the memory 223 may store the safe mode parameters used to switch the active parameters of the IMD 100 prior to a medical procedure. In another example, the memory 223 may store data indicative of cardiac signal waveforms, the fusion thresholds, predetermined time periods, fusion beat counts, total beat counts, and the like.

The safe mode parameters of the IMD 100 may be non-invasively programmed into the memory 223 through a telemetry circuit 264 in communication with the external device 252, such as a trans-telephonic transceiver or a diagnostic system analyzer. For example, the external device that telemetry circuit communicates with may be a non-programming activation device. The telemetry circuit 264 is activated by the microcontroller 220 by a control signal 266. The telemetry circuit 264 allows intra-cardiac electrograms, cardiac waveforms of interest, detection thresholds, status information relating to the operation of IMD 100, and the like, to be sent to the external device 252 through an established communication link 268.

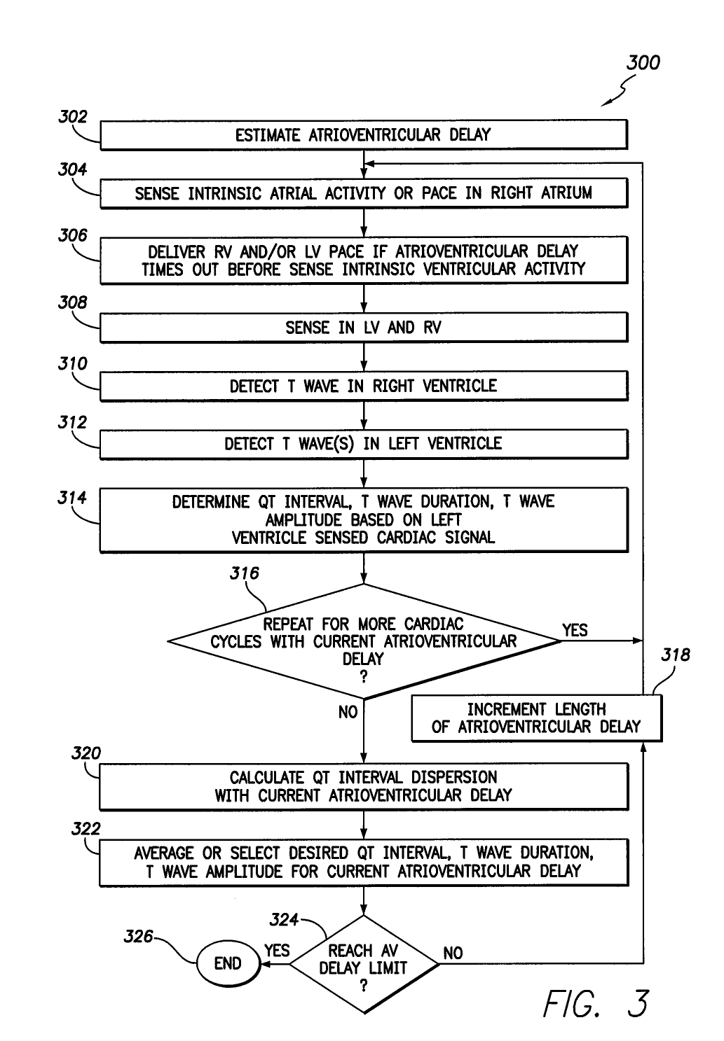

FIGS. 3-9 illustrate processes to improve CRT and pacing parameters, such as CRT AV timing, CRT VV timing, pacemaker AV timing, pacemaker VV timing and the like. The AV and/or VV timing may be set to a preferred level (e.g., optimized). The methods and processes described herein may be implemented by an IMD, an external programmer, a server, a distributed system and the like.

FIG. 3 illustrates a process to collect waveform metrics utilizing a T-wave based AV delay in accordance with an embodiment. The AV delay may represent an AV sense delay (e.g., sense P-wave and wait a time out before pace ventricle) and/or an AV paced delay (e.g., pace in the atrium and wait a time out before pacing in ventricle). The process 300 starts at 302, where an estimate is made for the AV delay. The AV delay may be chosen using one of several conventional methods of choosing AV delay. For example, the AV delay may be calculated using the QuickOpt algorithm from St. Jude Medical that provides IEGM-based AV interval and V-V timing determination in CRT devices.

At 304, the process senses intrinsic atrial activity or a pacing stimulus in the RA. The sensing operation may be done locally using RA lead 106. Alternatively, sensing may be done globally using a combination of RV lead 104, RA lead 106, and LV lead 108.

At 306, one or more ventricular pacing stimulus is/are delivered (LV, RV, or both) if the AV delay timer reaches a limit (e.g., times out) before sensing intrinsic ventricular activity. At 306, one or multiple stimulus may be delivered in a single ventricular chamber, or in both ventricular chambers. Flow moves to 308 from 306 without a pacing stimulus, when an intrinsic event is detected in the RV or LV before the AV delay times out. At 308, the process senses cardiac signal in the RV and the LV to obtain global activation information. The delay in the normal flow of electrical impulses from an atrium to a ventricle may be caused by, inter alia, a heart block. For example, a heart block that occurs above the AV node may be caused by the SA nodal block or the AV nodal block.

At 310, the process detects a T-wave in the cardiac signal sensed at the RV electrode following the RA paced or intrinsic activity. At 312, the method detects a T-wave in the cardiac signal sensed at one or more LV electrodes. The T-wave may be detected on each electrode combination utilized in the LV. The timing of the T-wave may be measured in different manners based on various features of interest. For example, the peak of the T-wave may be designated as the feature of interest in the T-wave. Alternatively, the feature of interest that defines the timing of the T-wave may be the last time point at which the T-wave goes below a predetermined threshold during a cardiac cycle. Another possible feature of interest, for identifying the timing of the T-wave, may be finding the T-wave midpoint, based on the center of mass of the T-wave. Another feature of interest, for identifying the T-wave, may be tracking the time at which the T-wave exhibits a minimum value of a negative deflection.

At 314, the process determines one or more waveform metrics, such as the QT interval based on the LV sensed cardiac signals. For example, the QT interval is a measure of the time between the start of the Q wave and the end of the T-wave in the heart's electrical cycle. The QT interval can be measured by determining onset of the QRS complex and the end of the T-wave. The onset of the QRS complex may be defined as the initial downward deflection of the QRS complex. The relative contribution of the beginning of the QT interval to overall QT variability is small as compared to the T-wave

Optionally, at 314, the process may determine the T-wave duration (as another waveform metric) from the LV sensed cardiac signals. The T-wave duration may be measured by calculating the time difference between the start and the end of the T-wave. For example, the start and the end times of the T-wave may be determined by using a threshold method. Alternatively, the start and the end times of the T-wave may be determined by using a tangential method.

Optionally, at 314, the process may determine the T-wave amplitude (as another waveform metric) from the LV sensed cardiac signals. The T-wave amplitude may be measured as peak-to-peak amplitude, peak amplitude, semi-amplitude, or root mean square amplitude and the like. The peak-to-peak amplitude may be measured as the change between peak (highest amplitude value) and trough (lowest amplitude value, which can be negative) of the T-wave. Peak amplitude may be measured as a maximum deflection in magnitude of the T-wave from the isoelectric base line. The semi-amplitude of the T-wave may be measured as half the peak-to-peak amplitude. The root mean square amplitude may be measured as the square root of the arithmetic mean (average) of the squares of the peak T-wave amplitude.

At 316, the process determines based on stored parameters whether the operations at 304-314 are to be repeated for multiple cardiac cycles or for additional electrode combinations utilizing the current AV delay. For example, it may be desirable to collect QT intervals and other waveform metrics for a series of cardiac cycles while utilizing the current AV delay to improve resolution of time measurements. It may also be desirable to collect QT intervals and other waveform metrics for more electrode combinations than can be measured at a time. The number of cardiac cycles in the series, over which data is collected, may be programmed or automatically updated. If the decision is “yes”, the flow moves to 304 and the process is repeated again. If the decision is “no”, the flow moves to 320.

At 320, a QT interval dispersion is calculated from the QT intervals measured at each of the electrode combinations, and may also take into account the difference in QT intervals measured at individual electrode combinations over one or a sequence of cardiac cycles. The QT interval dispersion may provide temporal dispersion information. For one electrode configuration over multiple cardiac cycles. Optionally, the QT interval dispersion may provide spatial dispersion information for different electrode configurations either i) during the same cardiac cycle or ii) over successive cardiac cycles while maintaining pacing parameters constant. For example, the QT interval may be measured as the interval between initial downward deflection of the QRS complex and the end of the T-wave. The end of the T-wave may be measured as the T-wave goes below a threshold or as the T-wave merges with the isoelectric baseline.

At 322, a desired one of the waveform metrics is selected from one cardiac cycle, or from a series of cardiac cycles. For example, a desired one of the QT intervals may be selected. Alternatively, a desired one of the T-wave duration or one of the T-wave amplitudes may be selected. Alternatively, at 322 the desired QT interval, T-wave duration and/or T-wave amplitude may be delivered using average values of the corresponding parameter over multiple cardiac cycle.

At 324, it is determined whether the current AV delay has reached an AV delay limit. If the answer is “No”, the flow moves to 318. At 318, the current AV delay is incremented and the flow moves back to step 304 where the process of 304 to 322 is repeated for a new series of cardiac cycles with a new AV delay. The operations at 304 to 322 are repeated for multiple AV delays to obtain multiple values for the waveform metric or metrics of interest. Returning to 324, if the current AV delay has reached the AV delay limit, the process of FIG. 3 ends 326.

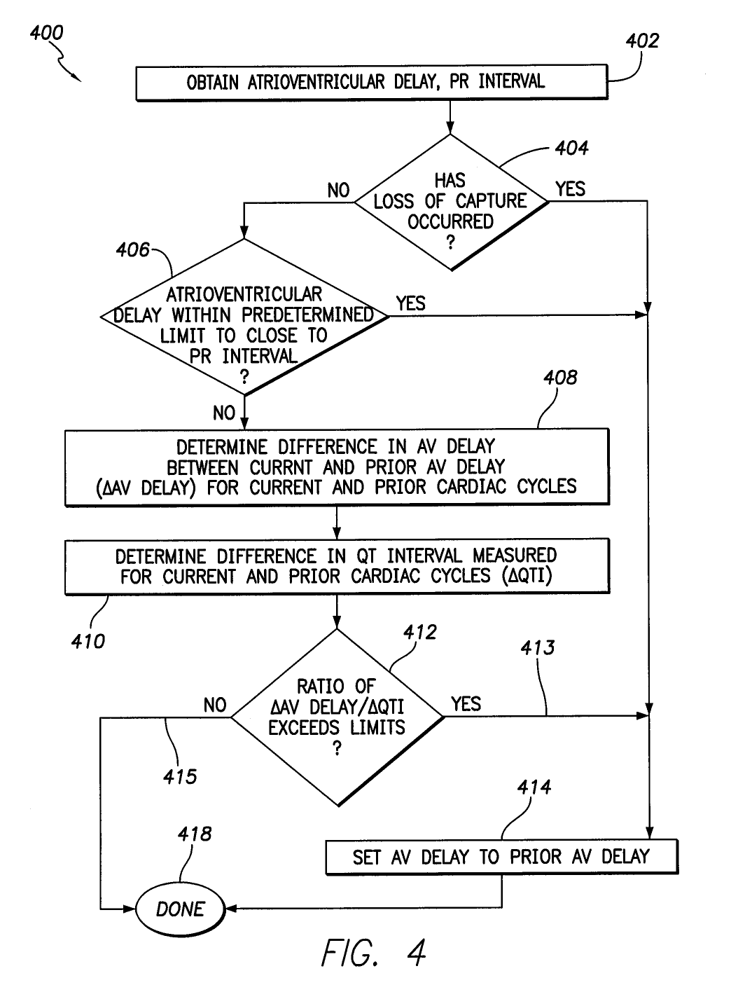

FIG. 4 illustrates a process 400 to determine whether the current AV delay has reached the AV delay limit (at 324 in FIG. 3) in accordance with an embodiment. The flow starts, at 402, with obtaining the current AV delay and the PR interval on the currently sensed cardiac cycle. The PR interval may be programmed or modified by a physician at the time of implant or any time thereafter. Optionally, the IMD may automatically update the PR interval at certain times based on select criteria. At 404, the process checks to see if loss of capture has occurred. The IMD periodically performs a test for loss of capture. At 404, the results of the most recent test or series of tests for loss of capture are analyzed. Failure to capture or loss of capture happens when the stimulus output fails to initiate a depolarization process of the atrium/ventricle. For example, a loss of capture may occur when a paced stimulus is delivered while heart tissue is in a refractory state. If there is a loss of capture, the flow moves to 414. At 414, the current AV delay is shortened and set to the prior AV delay. The prior AV delay corresponds to the prior loop through the process of FIG. 3 when capture was achieved. After setting the current AV delay to the prior AV delay, the flow ends at 418. Returning to 404, when there is no loss of capture, the flow moves to 406.

At 406, the process performs a check to determine if the current AV delay is within a predetermined limit of the PR interval. It may be desirable to maintain the AV delay shorter than the PR interval. For example, the AV delay may be programmed to a predetermined amount of time, or to a percentage of the PR interval, and the like. If the current AV delay is too close to the PR interval, then flow moves to 414. At 414, the current AV delay is shortened and set to the prior AV delay. Following the setting of the AV delay, the flow ends at 418. Returning to 406, if the current AV delay is not too close to the PR interval, then the flow moves to 408.

At 408, a ΔAV delay is calculated as a difference between the current AV delay and the prior AV delay corresponding to the current series and prior series of the cardiac cycles, respectively. At 410, a ΔQT interval (ΔQTI) is calculated as a difference between the current and prior. QT intervals associated with current series and prior series of the cardiac cycles, respectively.

At 412, the process checks whether the ratio of ΔAV delay to the ΔQTI exceeds a predetermined limit. If the ratio of the ΔAV delay to the ΔQTI exceeds a predetermined limit, then flow moves along 413 to 414. Returning to 412, if the ratio of the ΔAV delay to the ΔQTI does not exceed the predetermined limit, flow moves along 415. At 418, flow returns to 324 in FIG. 3 and from 324 flow moves to 318 where the AV delay is incremented for the next iteration through the process of FIG. 3.

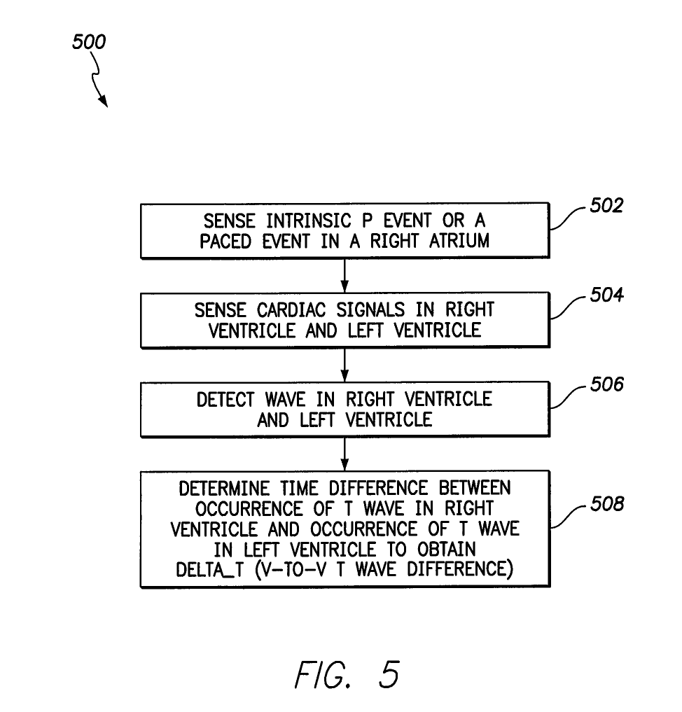

FIG. 5 illustrates a process to collect interval related information following a ventricular paced event in accordance with an embodiment. The interval related information represents the ventricle-to-ventricle difference in T-wave timing in accordance with an embodiment. The process 500 starts by tracking the beginning of a new cardiac cycle. The new cardiac cycle may begin with an intrinsic P-wave or a paced atrial event. At 502, the process senses an intrinsic P event or a pacing stimulus in the RA. At 504, following the detection of the intrinsic P-wave or pacing stimulus, the IMD begins listening to the cardiac signals in the RV and in the LV. Also at 504, the RV and the LV cardiac signals are collected for the cardiac cycle associated with the intrinsic P-wave or pacing stimulus from 502.

At 506, the process analyzes the RV and the LV cardiac signals to detect the times of the T-wave in each of the RV and the LV cardiac signals. By way of example, the timing of the T-wave may be determined as discussed above.

At 508, the process determines a time difference between the occurrence of the T-wave sensed in the RV and the occurrence of the T-wave sensed in the LV. The difference, between the occurrences of the T-wave, called Delta_T, represents a V-to-V T-wave difference. By way of example, the midpoint of the T-wave from the RV cardiac signal may occur 30 msec after an intrinsic P-wave event, while the midpoint of the T-wave from the LV cardiac signal may occur 50 msec after the intrinsic P-wave event. Hence, the Delta_T would be 20 msec. As another example, the peak of the T-wave in the LV cardiac signal may occur 10 msec after a paced atrial event, while the peak of the T-wave in the RV cardiac signal occurs 40 msec after the paced atrial event. Hence, the Delta_T would be 30 msec. The Delta_T is utilized, as explained below, to calculate various pacing parameters.

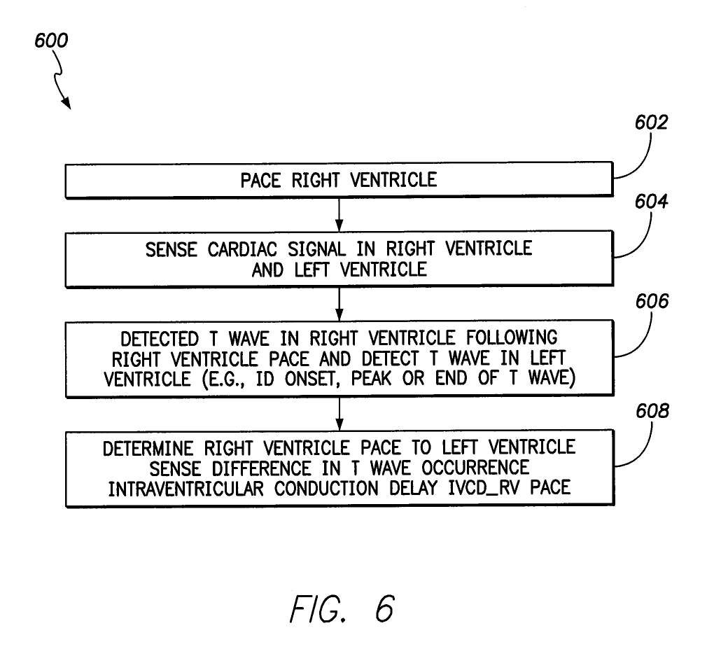

FIG. 6 illustrates a process 600 to determine a ventricular conduction delay from the RV to the LV in accordance with an embodiment. The flow starts at 602 with the IMD delivering a pacing stimulus in the RV. At 604, the process senses cardiac signals in the RV and in the LV. At 606, the process analyzes the sensed RV cardiac signal to detect the T-wave in the RV following the RV pacing stimulus. Also, at 606, the process analyzes the LV cardiac signal to detect the T-wave in the LV following the RV pacing stimulus. At 608, the process determines the ventricular conduction delay (IVCD_RV pace) by calculating the difference in the timing of the T-wave occurrence between the RV and the LV following the RV pacing stimulus. By way of example, minimum negative deflection of the T-wave in the RV may occur 210 msec after the RV pacing stimulus, while the minimum negative deflection of the T-wave in the LV may occur 225 msec after the RV pacing stimulus. Hence, the IVCD_RV pace following an RV pacing stimulus would be 15 msec.

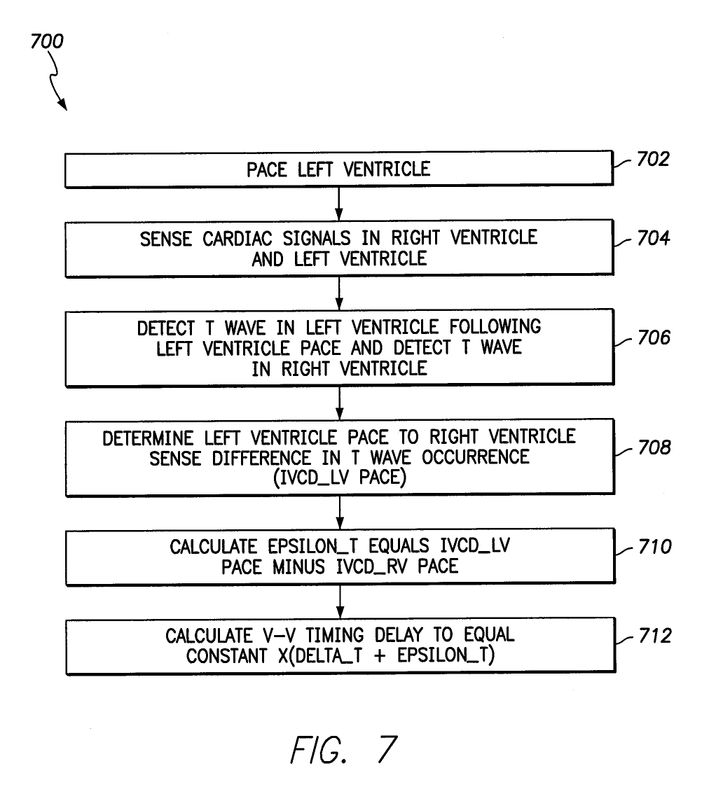

FIG. 7 illustrates a process to calculate a ventricular conduction delay following an LV pacing stimulus and a process to calculate a V-V interval in accordance with an embodiment. The flow starts at 702 with a pacing stimulus in the LV. At 704, the process senses cardiac signals in the RV and in the LV following pacing in the LV. At 706, the process detects the T-wave in the LV sensed cardiac signal following the LV pacing stimulus. Also, at 706 the process detects the T-wave in the RV sensed cardiac signal following the LV pacing stimulus. At 708, the process calculates the difference in the time of occurrence of the T-wave in the RV and the time of occurrence of the T-wave in the LV, both following the LV pacing stimulus. The difference between the times of occurrence of the T-wave in the LV and the T-wave in the RV represents the ventricular conduction delay following an LV pacing stimulus, also denoted as IVCD_LV pace.

At 710, the process calculates Epsilon_T, where Epsilon_T is the difference between IVCD_LV pace and IVCD_RV pace. The value of Epsilon_T represents a directional temporal difference in inter-ventricular conduction delays based on a direction of propagation, namely from the RV to the LV or from the LV to the RV.

At 712, a V-V timing delay is calculated as a product of a constant and the sum of Delta_T and Epsilon_T, namely the V-V timing delay=Constant×(Delta_T+Epsilon_T).

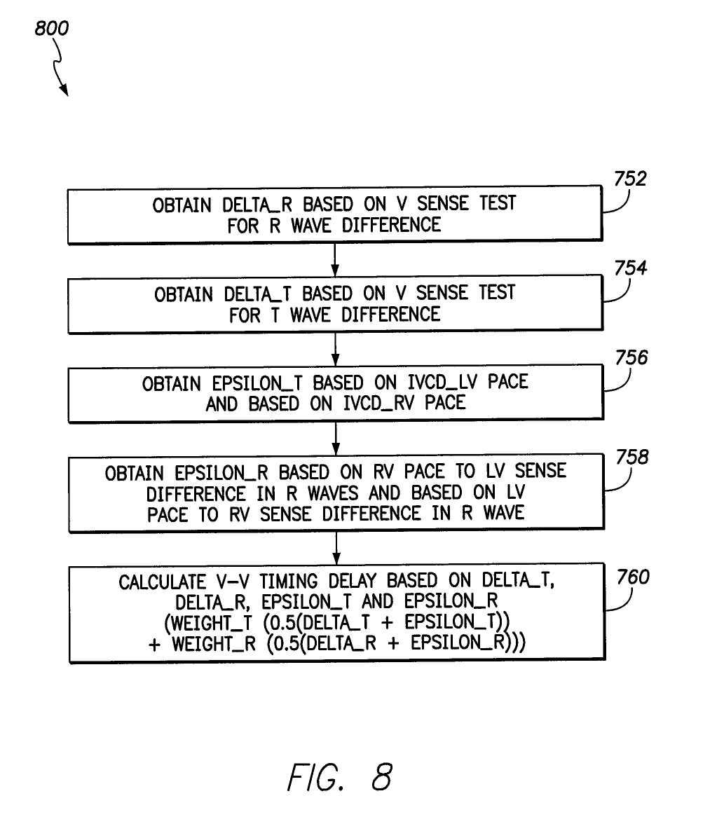

FIG. 8 illustrates an alternate process 800 to calculate a V-V interval based on the timing of the T-wave and based on the timing of the R-wave in accordance with an embodiment. In FIG. 8, the processes of FIGS. 3-7 are repeated, and values are obtained for Delta_T, Epsilon_T, IVCD_RV pace and IVCD_LV pace based on features of interest form the T-wave as explained above. In addition, the cardiac signals are analyzed based on features of interest from the R-wave, to determine values for Delta_R, Epsilon_R, IVCD_RV pace and IVCD_LV pace.

At 752, the process obtains Delta_R based on a ventricular sensing test that determines a difference in the time of occurrence of an R-wave as measured in the RV and the time of occurrence of an R-wave as measured in the LV following an intrinsic P-wave or atrial paced event. More specifically, at 752, the IMD 100 senses cardiac signals in the RV and in the LV. The process detects an R-wave in each of the RV and LV cardiac signals. Based on the detected R-waves in the RV and LV cardiac signals, the process determines a time difference between the occurrence of the R-wave sensed in the RV and the occurrence of the R-wave sensed in the LV. The difference between the occurrences of the R-waves, called Delta_R, represents a V-to-V R wave difference.

At 754, the process determines a time difference between the occurrence of the T wave sensed in the RV and the occurrence of the T-wave sensed in the LV (Delta_T). The complete process for obtaining the Delta_T is illustrated in FIG. 5.

At 756, the Epsilon_T is calculated based on the IVCD_LV pace and the IVCD_RV pace values. The process to calculate Epsilon_T is described in FIG. 6 and FIG. 7 supra. The value of Epsilon_T is calculated as the difference between the value of the IVCD_LV pace and the value of the IVCD_RV pace (Epsilon_T=IVCD_LV pace−IVCD_RV pace).

At 758, the process of FIGS. 6 and 7 is repeated to obtain a value for Epsilon_R. Epsilon_R is denoted ad the difference between R_IVCD_LV pace and R_IVCD_RV pace. The R_IVCD_RV pace represents the occurrence of the R-wave as sensed in the LV based on a RV pacing stimulus (R_IVCD_RV pace). R_IVCD_LV pace represents the occurrence of the R-wave as sensed in the RV based on a LV pacing stimulus (R_IVCD_RV pace). The process to calculate the R_IVCD_LV pace starts with the LV pacing stimulus. The process then senses the cardiac signal in the RV and the LV following the LV pacing stimulus. The process analyses the sensed LV cardiac signal to detect the R-wave. Also, the process analyses the sensed RV cardiac signal to detect the R-wave. The process then calculates the time difference in the occurrence of the R-wave in the RV and in the LV following the LV pacing stimulus to obtain R_IVCD_LV pace.

The process to calculate the R_IVCD_RV pace starts with the RV pacing stimulus. Cardiac signals are sensed in the RV and the LV following the RV pacing stimulus. The sensed RV cardiac signal is analyzed to detect the R-wave. The sensed LV cardiac signal is analyzed to detect the R-wave. The process then calculates the time difference in the occurrence of the R-wave in the RV and in the LV following the RV pacing stimulus to obtain R_IVCD_RV pace.

At 760, the process determines the value of V-V timing delay also referred to as the inter-ventricular interval. The value of V-V timing delay is the sum of R-wave based V-V timing and the T-wave based V-V timing. R-wave based V-V timing is calculated as 0.5(Delta_R+Epsilon_R). Where Delta_R is the time difference between the occurrence of the R-wave sensed in the RV and the occurrence of the R-wave sensed in the LV denotes. Also, the value of the Epsilon_R is calculated as the difference between the value of R_IVCD_LV pace and the value of R_IVCD_RV pace. As noted above, the variable R_IVCD_LV pace is calculated as the time difference in the occurrence of the R-wave in the RV and in the LV following the LV pacing stimulus. The variable R_IVCD_RV pace is calculated as the time difference in the occurrence of the R-wave in the RV and in the LV following the RV pacing stimulus.

T-wave based V-V timing is calculated as 0.5(Delta_T+Epsilon_T). Where Delta_T is the time difference between the occurrence of the T-wave sensed in the RV and the occurrence of the T-wave sensed in the LV. The value of the Epsilon_T is calculated as the difference between the value of the IVCD_LV pace and the value of the IVCD_RV pace. As noted above, the variable IVCD_LV pace is calculated as the time difference in the occurrence of the T-wave in the RV and in the LV following the LV pacing stimulus. The variable IVCD_RV pace is calculated as the time difference in the occurrence of the T-wave in the RV and in the LV following the RV pacing stimulus.

Thus V-V timing delay is calculated as V-V timing delay=(Weight_T×(0.5×(Delta_T+Epsilon_T))+Weight_R×(0.5(Delta_R+Epsilon_R))). Where the Weight_T and the Weight_R are constants that a user may set to change the importance of the R-wave based V-V timing and the T-wave based V-V timing. For example, the Weight_T could be 0.40 and the Weight_R could be 0.60.

Optionally, the process of FIGS. 5-8 may be implemented in connection with setting an intra-ventricular interval which represents a time following a paced or intrinsic event before delivering a pacing stimulus in the same ventricular chamber.

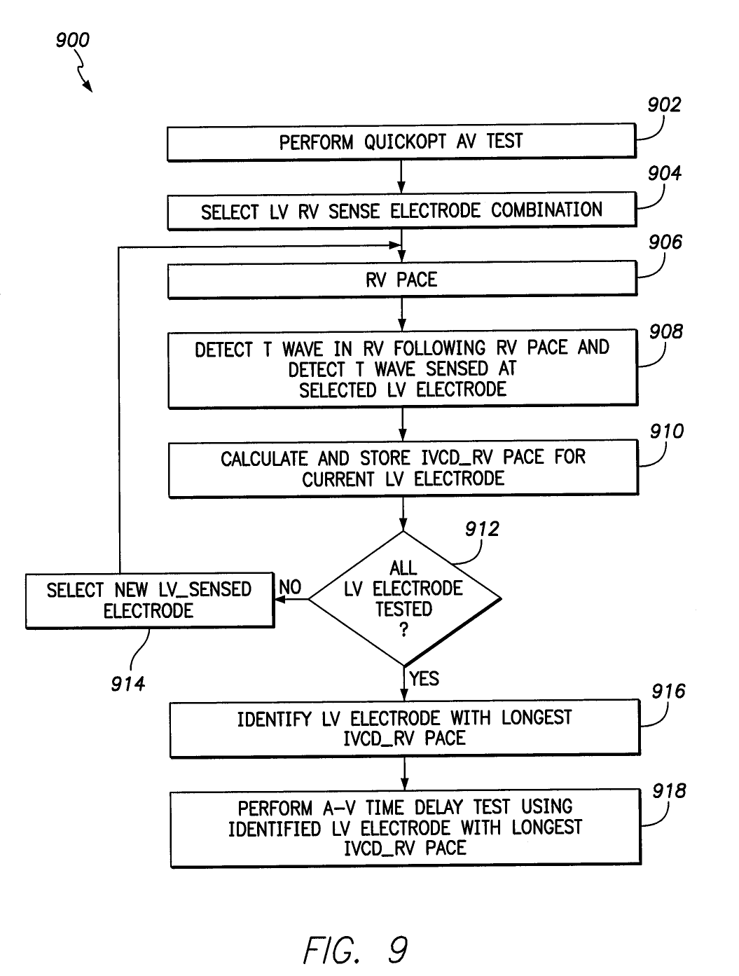

FIG. 9 illustrates an alternate process to perform an AV delay extension test utilizing a multipolar LV lead in accordance with an embodiment. The process 900 involves builds on the processes discussed above for estimation of the AV delay. At 902, for example, an offset may be added to the width of the intrinsic P-wave or atrial evoked response to determine the desired AV delay. At 904, the process involves selecting one of the LV sensing electrode from the multipolar LV lead. At 906, a pacing stimulus is delivered in the RV. The RV pacing stimulus is delivered at an estimated AV delay, obtained at flow 902. At 908, a T-wave is detected in the RV following the RV pacing stimulus. Also at 908, a T-wave is detected in the LV following the RV pacing stimulus. At 910, the process calculates and stores an inter-ventricular conduction delay (IVCD_RV pace). The variable IVCD_RV pace is calculated as the time difference in the occurrence of the T-wave in the RV and in the LV following the RV pacing stimulus.

At 912, it is determined whether all of the potential LV electrodes have been utilized to acquire LV cardiac signals and derive corresponding conduction delays. For example, if the LV lead has four LV electrodes, the operations from 906 to 910 may be repeated four times. The operation from 906 to 910 is repeated with a new LV electrode 914. Returning to flow at 912, when all the desired LV electrodes have been utilized to acquire LV cardiac signals, the flow moves to 916.

At 916, the process involves identifying the LV electrode with the longest IVCD_RV pace interval. The process compares the recorded IVCD_RV pace intervals associated with each of the LV electrodes. For example, a user may do the comparison manually to select the longest IVCD_RV pace interval. Alternatively, the IMD or external programmer may be configured to perform the comparison of all the recorded IVCD_RV pace intervals and select the longest IVCD_RV pace interval. After the determination of the longest IVCD_RV pace interval, the flow moves to 918. At 918, the process performs an AV time delay test using the LV electrode with longest IVCD_RV pace interval.

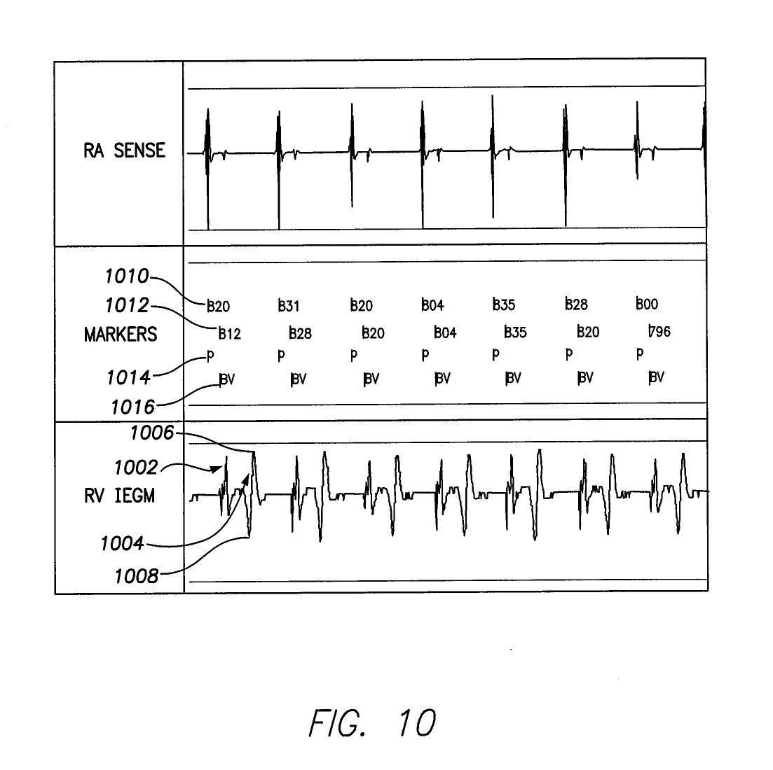

FIG. 10 illustrates a RA sensed intracardiac electrogram, and an RV sensed intracardiac electrogram along with markers in accordance with an embodiment. The RA and the RV waveforms are sensed following biventricular pacing. However, the LV waveform is not shown. RV IEGM waveform shows repeat cardiac cycles displaying the R-wave 1002 followed by the T-wave 1004. The T-wave, in RV IEGM also shows the repolarization spike 1006, the point of maximum amplitude during repolarization, and the minimum negative point 1008, the point of minimum amplitude during repolarization, of the T-wave.

The markers shown in FIG. 10 illustrate activity performed during the RA and the RV IEGM. Reference numeral 1010 denotes a row of the markers to show the time, in msec, at which the R-wave is recorded in the RA IEGM. The reference numeral 1012 denotes a row of markers shows the time, in msec, at which the T-wave is recorded in the RA IEGM. The reference numeral 1014 indicates a row of markers showing the time of an atrial sensing event and the reference numeral 1016 denotes a row of markers showing the time of a bi-ventricular paced event.

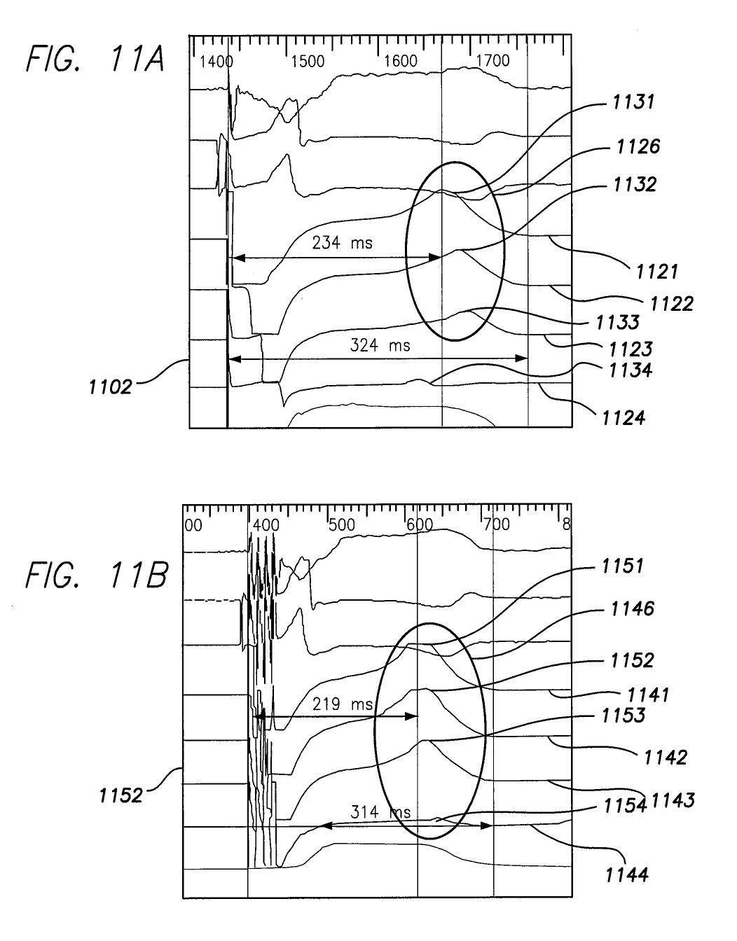

FIG. 11 illustrates exemplary cardiac signals sensed using a quadpolar LV lead in accordance with an embodiment. FIG. 11(a) illustrates high repolarization dispersion with single-site pacing for the LV tip. The circled portion shows dispersion in T-waves for the three electrodes. FIG. 11(b) illustrates low repolarization dispersion with all four LV electrodes paced nearly simultaneously.

In FIG. 11a, cardiac signals 1121-1124 are illustrated representative of cardiac signals sensed from four separate LV electrodes. The area generally denoted at 1126 represents the general areas of the T-waves within each of the cardiac signals 1121-1124. The T-waves within the cardiac signals 1121-1124 exhibit different timings, such as the point in time at which each T-wave reaches a peak, the point in time corresponding to the midpoint or center of mass of each T-wave, or the point in time in which each T-wave crosses a predetermined threshold. In the example of FIG. 11a, the peaks 1131-1134 are offset from one another in time.

FIG. 11b illustrates cardiac signals 1141-1144 sensed by corresponding LV electrodes. The area denoted at 1146 generally identifies the areas in which the T-waves occur within the cardiac signals 1141-1144. In the example of FIG. 11b, the timing or peaks of the T-waves 1151-1154 are generally temporally aligned in time with one another.

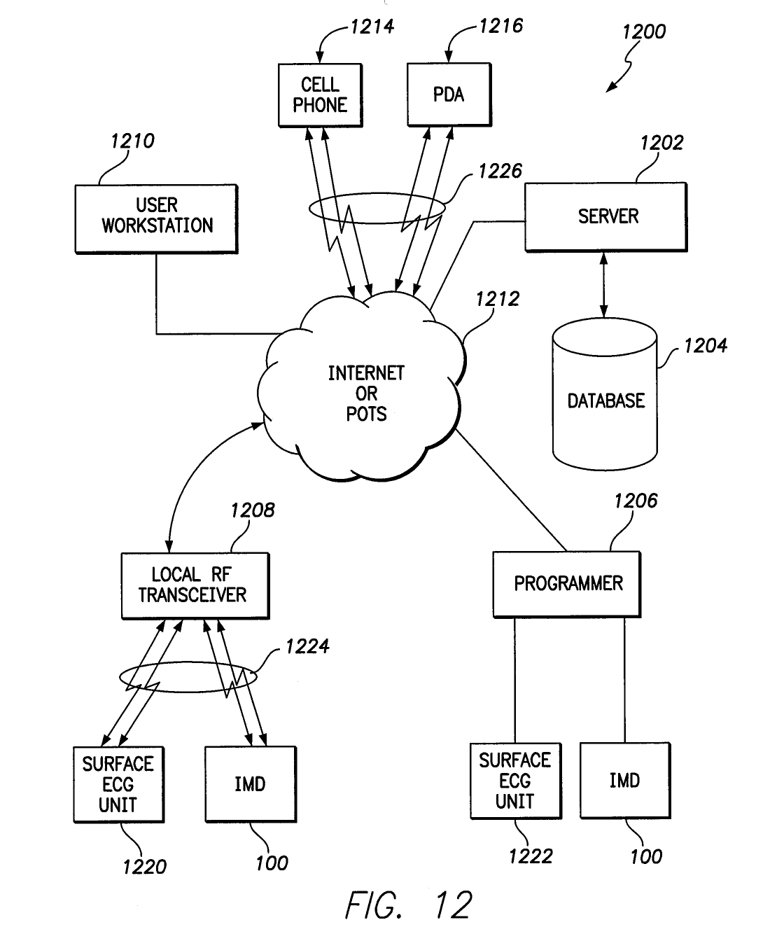

FIG. 12 illustrates a distributed processing system in accordance with an embodiment. The distributed processing system 1200 includes a server 1202 connected to a database 1204, a programmer 1206 (e.g., similar to external device 252 (shown in FIG. 2)), a local RF transceiver 1208, and a user workstation 1210 electrically connected to a communication system 1212. The communication system 1212 may be the internet, a voice over IP (VoIP) gateway, a local plain old telephone service (POTS) such as a public switched telephone network (PSTN), a cellular phone based network, and the like. Alternatively, the communication system 1212 may be a local area network (LAN), a campus area network (CAN), a metropolitan area network (MAN), or a wide area network (WAN). The communication system 1212 serves to provide a network that facilitates the transfer/receipt of information such as cardiac signal waveforms, fusion thresholds, fusion beat counts, total beat counts, IMD 100 programming data, safe mode signal to the IMD 100, and the like.

The server 1202 is a computer system that provides services to other computing systems over a computer network. The server 1202 controls the communication of information such as cardiac signal waveforms, fusion thresholds, fusion beat counts, total beat counts, and the like. The server 1202 interfaces with the communication system 1212 to transfer information between the programmer 1206, the local RF transceiver 1208, the user workstation 1210 as well as a cell phone 1214 and a personal data assistant (PDA) 1216 to the database 1204 for storage/retrieval of records of information.

The database 1204 stores information such as cardiac signal waveforms, fusion thresholds, fusion beat counts, total beat counts, IMD 100 programming data, safe mode signal to the IMD 100, and the like, for a single or multiple patients. The information is downloaded into the database 1204 via the server 1202 or, alternatively, the information is uploaded to the server from the database 1204. The programmer 1206 may reside in a patient's home, a hospital, or a physician's office. The programmer 1206 interfaces with the surface ECG unit 1222 and the IMD 100. The programmer 1206 may wirelessly communicate with the IMD 100 and utilize protocols, such as Bluetooth, ZigBee/802.15.4, GSM, infrared wireless LANs, HIPERLAN, 3G, satellite, as well as circuit and packet data protocols, and the like. Alternatively, a hard-wired connection may be used to connect the programmer 1206 to the IMD 100. The programmer 1206 is able to acquire cardiac signals from the surface of a person (e.g., ECGs), intra-cardiac electrogram (e.g., IEGM) signals from the IMD 100, and/or cardiac signal waveforms, fusion thresholds, fusion beat counts, total beat counts, and the like, from the IMD 100. The programmer 1206 interfaces with the communication system 1212, either via the internet or via POTS, to upload the information acquired from the surface ECG unit 1220 or the IMD 100 to the server 1202.

The local RF transceiver 1208 interfaces with the communication system 1212 to upload one or more of cardiac signal waveforms, ventricular and atrial heart rates, and detection thresholds to the server 1202. In one embodiment, the surface ECG unit 1220 and the IMD 100 have a bi-directional connection 1224 with the local RF transceiver 1208 via a wireless connection. The local RF transceiver 1208 is able to acquire cardiac signals from the surface of a person, intra-cardiac electrogram signals from the IMD 100, and/or cardiac signal waveforms, fusion thresholds, fusion beat counts, total beat counts, and the like, from the IMD 100. On the other hand, the local RF transceiver 1208 may download stored cardiac signal waveforms, fusion thresholds, fusion beat counts, total beat counts, and the like, from the database 1204 to the surface ECG unit 1220 or the IMD 100.

The user workstation 1210 may interface with the communication system 1212 via the internet or POTS to download cardiac signal waveforms, fusion thresholds, fusion beat counts, total beat counts, NPA unit log and the like via the server 1202 from the database 1204. Alternatively, the user workstation 1210 may download raw data from the IMD 100 via either the programmer 1206 or the local RF transceiver 1208. The user workstation 1210 may download the information and notifications to the cell phone 1214, the PDA 1216, the local RF transceiver 1208, the programmer 1206, or to the server 1202 to be stored on the database 1204.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the subject matter disclosed herein without departing from its scope. While the dimensions, types of materials and coatings described herein are intended to define the parameters of the subject matter disclosed herein, they are by no means limiting and are exemplary embodiments. Many other embodiments will be apparent to those of skill in the art upon reviewing the above description. The scope of the subject matter disclosed herein should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. §112, sixth paragraph, unless and until such claim limitations expressly use the phrase “means for” followed by a statement of function void of further structure.

What is claimed is:

1. A method for determining at least one pacing parameter for an implantable medical device (IMD), the method comprising:
providing leads to be located in a right ventricle (RV) and left ventricle (LV);
sensing RV cardiac signals and LV cardiac signals at an RV electrode and an LV electrode;
identifying a T-wave in the LV cardiac signal;
calculating a repolarization index based at least in part on a timing of the T-wave identified in the LV cardiac signal; and
setting at least one pacing parameter based on the repolarization index, wherein the at least one pacing parameter that is set represents at least one of an AV delay, an inter-ventricular interval and an intra-ventricular interval.

2. The method of claim 1, further comprising:
delivering an RV pacing stimulus at the electrodes of an RV lead such that the LV cardiac signal sensed thereafter includes a response to the RV pacing stimulus followed by a T-wave;
determining at least one of a QT interval, T-wave duration, and T-wave amplitude, as the repolarization index, based, at least in part, on the T-wave identified in the LV cardiac signals.

3. The method of claim 2, wherein the delivering and determining operations are repeated for multiple cardiac cycles, further comprising:
adjusting an AV delay between the multiple cardiac cycles;
collecting QT intervals for the multiple cardiac cycles; and
determining a dispersion of the QT interval.

4. The method of claim 1, further comprising:
measuring times of occurrence of T-waves in the LV cardiac signal and in the RV cardiac signal; and
determining a difference between the times of occurrence, the repolarization index being calculated at least in part based on the difference between the times of occurrence.

5. The method of claim 1, further comprising:
delivering an RV pacing stimulus at electrodes of the RV lead;
performing the sensing operation after delivering the RV pacing stimulus;
identifying a T-wave in the RV cardiac signal; and
calculating a ventricular conduction delay (IVCD_RVpace) as a time between occurrence of the T-wave in the RV cardiac signal and a time of occurrence of the T-wave in the LV cardiac signal, the IVCD_RVpace being used to calculate the repolarization index to then set the at least one pacing parameter.

6. The method of claim 1, further comprising:
delivering an LV pacing stimulus at one or more electrodes of an LV lead electrode;
performing the sensing operation after delivering the LV pacing stimulus;
identifying a T-wave in the RV cardiac signal; and
calculating a ventricular conduction delay (IVCD_LVpace) as a time between occurrence of the T-wave in the RV cardiac signal and a time of occurrence of the T-wave in the LV cardiac signal, the IVCD_LVpace being used to calculate the repolarization index to then set the at least one pacing parameter.

7. The method of claim 1, further comprising:
delivering an LV pacing stimulus at LV lead electrode and an RV pacing stimulus at the RV lead during different cardiac cycles; and
calculating first and second ventricular conduction delays based on the LV and RV pacing stimulus, the first and second ventricular conduction delays being used to calculate the repolarization index to then set the at least one pacing parameter.

8. The method of claim 7, further comprising setting the inter-ventricular interval based on the repolarization index.

9. The method of claim 1, wherein multiple LV electrodes are provided in or proximate to the LV, the method further comprising:
switching between different RV_LV combinations utilizing the RV electrode and different ones of the LV electrodes; and
repeating the sensing, identifying and calculating operations for the different RV_LV combinations.

10. The method of claim 9, further comprising:
identifying one of the RV_LV combinations that has a longest time between an occurrence of the T-wave in the RV cardiac signal and a time of occurrence of the T-wave in the LV cardiac signal; and
utilizing the identified one of the RV_LV combinations when performing an AV delay extension test.

11. The method of claim 1, wherein multiple LV electrodes are provided in or proximate to the LV, the method further comprising:
switching between different RV_LV combinations utilizing the RV electrode and the LV electrodes;
delivering LV pacing stimulus at the LV electrodes during different cardiac cycles;
detecting R-waves in the RV cardiac signals sensed at the RV electrode in connection with each of the LV pacing stimulus from the corresponding LV electrodes; and
identifying one of the RV_LV combinations that has an shortest time between an occurrence of the R-wave in the RV cardiac signal and a time of occurrence of the corresponding LV pacing stimulus.

12. A system for determining pacing parameter for an implantable medical device (IMD), the system comprising:
inputs to receive RV cardiac signals and LV cardiac signals that are sensed from the RV and the LV;
a processor to identify a T-wave in the LV cardiac signal;
the processor to calculate a repolarization index based at least in part on a timing of the T-wave identified in the LV cardiac signal; and
the processor to set at least one pacing parameter based on the repolarization index, wherein the at least one pacing parameter that is set represents at least one of an AV delay, an inter-ventricular interval and an intra-ventricular interval.

13. The system of claim 12, further comprising:
an RV electrode to deliver an RV pacing stimulus at the RV electrode such that the LV cardiac signal sensed thereafter includes the RV pacing stimulus followed by a T-wave;
the processor to determine at least one of a QT interval, T-wave duration, and T-wave amplitude, as the repolarization index, based, at least in part, on the T-wave identified in the LV cardiac signals.

14. The system of claim 12, wherein the RV and LV cardiac signals are sensed for multiple cardiac cycles, the processor adjusts an AV delay between the multiple cardiac cycles, the processor collects QT intervals for the multiple cardiac cycles; and the processor determines a dispersion of the QT interval for the multiple cardiac cycles.

15. The system of claim 12, wherein the processor measures times of occurrence of T-waves in the LV cardiac signal and in the RV cardiac signal; and determines a difference between the times of occurrence, the repolarization index being calculated at least in part based on the difference between the times of occurrence.

16. The system of claim 12, further comprising:
RV and LV electrodes, the RV electrode to deliver an RV pacing stimulus at the RV electrode, the RV and LV electrodes to sense the RV and LV cardiac signals after delivering the RV pacing stimulus, the processor to identify a T-wave in the RV cardiac signal; and the processor to calculate a ventricular conduction delay (IVCD_RVpace) as a time between occurrence of the T-wave in the RV cardiac signal and a time of occurrence of the T-wave in the LV cardiac signal, the IVCD_RVpace being used to calculate the repolarization index to then set the at least one pacing parameter.

17. The system of claim 12, further comprising:
RV and LV electrodes, the LV electrode to deliver an LV pacing stimulus at the LV electrode, the RV and LV electrodes to sense the RV and LV cardiac signals after delivering the LV pacing stimulus, the processor to identify a T-wave in the RV cardiac signal, the processor to calculate a ventricular conduction delay (IVCD_LVpace) as a time between occurrence of the T-wave in the RV cardiac signal and a time of occurrence of the T-wave in the LV cardiac signal, the IVCD_LVpace being used to calculate the repolarization index to then set the at least one pacing parameter.

18. The system of claim 12, further comprising:
RV and LV electrodes to deliver an RV pacing stimulus and an LV pacing stimulus during different cardiac cycles, the processor to calculate first and second ventricular conduction delays based on the LV and RV pacing stimulus, the processor to use the first and second ventricular conduction delays to calculate the repolarization index to then set the at least one pacing parameter.

19. The system of claim 12, wherein the processor sets the inter-ventricular interval based on the repolarization index.

20. The system of claim 12, further comprising:
an RV electrode;
multiple LV electrodes provided in or proximate to the LV; and
switches to switch between different RV_LV combinations utilizing the RV electrode and different ones of the LV electrodes, the processor to repeat the sensing, identifying and calculating operations for the different RV_LV combinations.

21. The system of claim 20, wherein the processor identifies one of the RV_LV combinations that has a longest time between an occurrence of the T-wave in the RV cardiac signal and a time of occurrence of the T-wave in the LV cardiac signal, the processor to utilize the identified one of the RV_LV combinations when performing an AV delay extension test.

22. The system of claim 12, further comprising inputs to receive at least one of RA cardiac signals and LA cardiac signals that are sensed from the RA and the LA.
